(19) United States
(12) Patent Application Publication
(10) Pub. No.: US 2008/0188398 A1
(43) Pub. Date: Aug. 7, 2008

(54) Gas Odorant Comprising a Cycloalkadiene

(75) Inventors: Urs Mueller, Duebendorf (CH); John Norman Short, L'Isle Adam (FR)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (CH)

(21) Appl. No.: 11/915,800

(22) PCT Filed: May 30, 2006
(86) PCT No.: PCT/CH06/00281

§ 371 Date: Dec. 19, 2007

(30) Foreign Application Priority Data
May 30, 2005 (EP) 05291161.7
Aug. 31, 2005 (EP) 05291807.5

(51) Int. Cl. A61K 8/31; C07C 13/18

(52) U.S. Cl. 512/22; 585/23

(57) ABSTRACT

The present invention refers to the use as gas odorant of cycloalkadienes of formula (I)

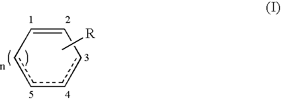

wherein R and n have the same meaning as given in the description, to a process of odorizing gas and to fuel gas comprising it.

The present invention relates to the use of cycloalkadienes as gas odorants, to a process of odorising gas and to fuel gas comprising them.

Because of their origin and their relative high degree of purity, fuel gases are substantially odorless. If leakages are not noticed in good time, explosive mixtures with a high hazard potential may be quickly formed. For safety reasons, gas is therefore odorized by adding odorants. For example, tetrahydrothiophene (THT) is used throughout the European Union area as a city gas odorant, usually as the sole odorant. In North America, tertiary butyl mercaptan is widely used as the principal odorant, often in association with other alkyl mercaptans and various sulphides and disulphides. For liquid petroleum gas, for example, ethyl mercaptan is used as an odorant. These compounds are particularly well suited for use as gas odorants because of their very powerful, unpleasant and distinctive odours. Furthermore, because they have been used for a very long time, they are nowadays universally associated with combustible gases and thus meet the requirement for a distinct and well recognized warning of a gas leakage. However, with regard to environmental aspects, sulfur compounds are less suitable because sulfur dioxide is formed during the combustion of such odorized gases.

To be highly suitable as gas odorant, a compound or a mixture of compounds, i.e. a composition, has to fulfill a number of requirements. In particular, to avoid the danger of confusion, the odour of the gas odorant needs to:
be distinctive enough that there is very little risk that it will be identified as any other smell. Ideally it should be immediately associated with the odour of current gas odorants, because their widespread use make the odour of gas easily recognizable to the majority of users.
have a detection threshold several orders of magnitude lower than the explosive limit of the fuel gas in order that anyone with an average sense of smell and average physiological conditions is able to detect the odor.

Furthermore, the gas odorant has to be stable under the storage and transport conditions of the fuel gas.

Several attempts have been made to replace or at least reduce the use of sulfur compounds as odorant in fuel gas. For example, DE-A 19837066 describes the use of a mixture of acrylic alkyl esters and nitrogen compounds. JP 2003-155488 A discloses more than 150 chemicals which are suitable as odorants for the addition to fuel hydrogen. According to the description to fulfill the requirement as odorant for fuel hydrogen is, that its odour can be clearly distinguished from a smell common in daily life, that it has a typical odour detectable at low concentration, and that it is harmless and non-toxic to humans. Further details, such as odor descriptions are not given. A fuel gas odorant comprising an alkyne such as but-1-yne, vinylacetylene and hexyne, and at least two compounds selected from methyl acrylate, ethyl acrylate, methyl methacrylate, allyl methacrylate, ethyl propionate, methyl n-butyrate, and methyl isobutyrate is disclosed in JP-A-55-104393 (abstract). The problem with acrylic alkyl esters is that their odor notes are very similar to, for example, certain acrylic plastics and paints.

Accordingly, there is an ongoing demand to find alternative odorants, which are suitable as gas odorants. For the replacement of current gas odorants, which have been used for a very long time it is particularly preferred that the substitutes have a gas like odor to be successfully introduced into the market.

Surprisingly, it has been found that cyclohexadienes and cyclooctadienes are particularly suitable as odorants for hydrocarbon gas due to their gas like odorant, whereas, for example, the structural related cycloheptadiens are much weaker and have a more green, citrusy and less gas like odour.

By gas like odorant is meant, that the odor of the compound immediately is associated with the odor of gas.

Accordingly, the present invention refers in one of its aspects to hydrocarbon gas comprising cycloalkadienes of formula (I) as gas odorant

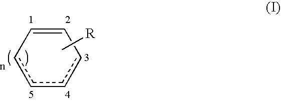

wherein

n is 1 or 3;

R is hydrogen or C₁-C₃ alkyl, e.g. methyl or isopropyl; and

if n is 1

the bond between C-3 and C-4 and C-5 and C-6 is a single bond and the dotted line together with the bond between C-4 and C-5 represents a double bond; or

the bond between C-4 and C-5 and C-5 and C-6 is a single bond and the dotted line together with the bond between C-3 and C-4 represents a double bond;

if n is 3

the bond between C-3 and C-4 and C-4 and C-5 is a single bond and the dotted line together with the bond between C-5 and C-6 represents a double bond; or

the bond between C-3 and C-4 and C-5 and C-6 is a single bond and the dotted line together with the bond between C-4 and C-5 represents a double bond; or

the bond between C-4 and C-5 and C-5 and C-6 is a single bond and the dotted line together with the bond between C-3 and C-4 represents a double bond;

and one carbon-carbon double bond is in Z configuration and the second carbon-carbon bond is in E or Z configuration, preferably both double bonds are in Z configuration.

Particularly preferred as a fuel gas odorant are 1,5-cyclooctadiene, (Z,Z)-1,5-cyclooctadiene, (Z,E)-1,5-cyclooctadiene, 3-methyl-1,5-cyclooctadiene, 1,3-cyclohexadiene, and 1,4-cyclohexadiene.

The term “gas odorant” as used within the meaning of this invention may refer to both a single odorous compound and a mixture of such odorous compounds.

Hydrocarbon gases are in general used for generating electrical power by combustion in power stations, or used in buildings for heating, illuminating and cooking processes. The term “hydrocarbon gas” as used within the meaning of this application stands for any combustible fuel gas used as a primary or secondary energy source. They are in gaseous form at normal atmospheric temperature and pressure (25° C.; 1000 mbar) but may also be processed in their liquid form for convenience of transport and storage. Fuel gases encompass, but are not limited to, the terms: city or town gas, natural gas including its liquefied form, and liquid petroleum gas (LPG, which is a mixture of alkanes separated from petrol and consisting essentially of butane and propane). Alkynes, such as acetylene, are also suitable as hydrocarbon gas. Oxygenated hydrocarbons, such as dimethyl ether also belong to the class of hydrocarbon gases within the meaning of this application.

The compounds of the present invention may be used alone or in combination with other gas odorants, i.e. with sulfur compounds and sulfur-free compounds. Particularly preferred is the combination with sulfur-free gas odorants, for example pyrazines, acrylic acid C₁-C₆ alkyl esters, alkynes such as 1-methoxy-buten-3-yne and 2-methyl-1-buten-3-yne, and cycloalkynes such as cyclooctyne, preferably in an amount of up to 10 parts by weight, more preferably 0.1 to 5.5 parts by weight per 100 parts by weight of an cycloalkadiene of formula (I) or a mixture thereof. If combined with sulfur compounds the fuel gas odorant preferably comprises up to 60 weight %, more preferably up to 30 weight %, e.g. 1 to 10 weight % of a sulfur compound or a mixture thereof, based on the total amount of gas odorant.

Suitable pyrazines include but are not limited to methyl ethyl pyrazine, methoxy isobutyl pyrazine, and methoxy methyl pyrazine. Further suitable pyrazines are disclosed in JP-A-08-60167 (U.S. Pat. No. 3,378,673), which is incorporated by reference. By admixing the compounds of the present invention together with a smaller amount of pyrazine even better results can be achieved.

Suitable acrylic acid C₁-C₆ alkyl esters include but are not limited to methyl acrylate, ethyl acrylate, n-propyl acrylate, iso-propyl acrylate, n-butyl acrylate, iso-butyl acrylate, tert-butyl acrylate, n-pentyl acrylate, iso-pentyl acrylate and n-hexyl acrylate.

Suitable sulfur compounds include but are not limited to compounds selected from the group consisting of C₁-C₄ alkyl mercaptan, e.g., tert-butyl mercaptan and ethyl mercaptan, aryl mercaptanes, e.g. benzyl mercaptan, organic sulfides and disulfides, e.g. dimethyl sulfide and ethyl methyl sulfide, and tetrahydrothiophene and their derivatives.

Accordingly, particularly preferred are gas odorants comprising:
a) at least one cycloalkadiene of formula (I); and
b) at least one sulfur-free compound selected from 1-methoxy-buten-3-yne, 2-methyl-1-buten-3-yne, cyclooctyne, pyrazines, wherein the pyrazine is preferably selected from the group consisting of methyl ethyl pyrazine, methoxy isobutyl pyrazine and methoxy methyl pyrazine, and acrylic acid C₁-C₆ alkyl esters; and/or
c) at least one sulfur compound.

Other additives such as antioxidants may also be added, either to the odorant or directly to the odorized fuel gas. Suitable antioxidants include but are not limited to tert. butylhdroxyanisole, 2,5-di-tert-butyl-phenol(lonol), hydroquinone monomethyl ether and α-tocopherol, 2,6-di-tert-butyl para cresol and tert-butyl hydroxy toluene.

A further aspect of the present invention is a hydrocarbon gas comprising a gas odorant comprising
a) at least one cycloalkadiene of formula (I);
b) and optionally at least one sulfur-free compound selected from 1-methoxy-buten-3-yne, 2-methyl-1-buten-3-yne, cyclooctyne and pyrazines, wherein the pyrazine is preferably selected from the group consisting of methyl ethyl pyrazine, methoxy isobutyl pyrazine and methoxy methyl pyrazine, and acrylic acid C₁-C₆ alkyl esters;
c) and optionally at least one sulfur compound.

The dosage of the gas odorant of the present invention in the hydrocarbon gas mainly depends on the composition of the odorant and may vary from 10 to about 200 ppm, preferably between 50 and 150 ppm, more preferably between 10 and 100 ppm.

Furthermore, the present invention refers to a method of odorizing hydrocarbon gas comprising the incorporation as odorant of an effective amount of at least one cycloalkadiene as hereinabove described.

The odorants of the present invention are liquids at room temperature and thus, both the preparation of an odorant composition if required and the admixing of the odorant/odorant composition to the hydrocarbon gas is not critical. The methods and equipments known to the person skilled in the art may be used.

The invention is now further described with reference to the following non-limiting examples.

### EXAMPLE 1

### Olfactive Evaluation

The compounds listed below have been prepared each at 150 ppm in an aerosol using isobutene/propane propellant and were sprayed into an evaluation both and sniffed through a small port in the door of the both.

| Compounds of formula (I) | Odor description |
|---|---|
| (Z,Z)-1,5-cyclooctadiene | gas like, sulfurous |
| 3-methyl-1,5-cyclooctadiene | gas like, sulfurous, onion, garlic |
| 1,3-cyclohexadiene | gas like, sulfurous, earthy, potato |
| 1,4-cyclohexadiene | gas like, sulfurous, earthy |

### EXAMPLE 2

### Gas Odorant Compositions

If the cycloalkadienes of formula (I), as defined herein above, are combined with other gas odorants the gas odorant compositions of Table 1 might be of particular interest.

TABLE 1

| | Composition | | | | | | |
|---|---|---|---|---|---|---|---|
| Compound | A | B | C | D | E | F | G |
| 1,5- | 95 | 98 | 95 | 97 | 91 | 96 | 90 |
| Cyclooctadiene | | | | | | | |
| Ethyl acrylate | 5 | — | — | — | 6 | — | 6 |
| | (5.26) | | | | (6.59) | | (6.67) |
| Methyl ethyl | — | 2 | — | — | — | — | — |
| pyrazine | | (2.04) | | | | | |
| 1-Methoxy- | — | — | 5 | — | — | — | — |
| buten-3-yne | | | (5.26) | | | | |
| tert-Butyl | — | — | — | 3 | 3 | — | — |
| mercaptan | | | | | | | |
| Dimethyl | — | — | — | — | — | 4 | 4 |
| sulfide | | | | | | | |

All amounts are given in weight % unless otherwise indicated. The numbers given in brackets refer to parts by weight per 100 parts by weight of 1,5-cyclooctadiene.

1. A hydrocarbon gas comprising a cycloalkadiene of formula (I) 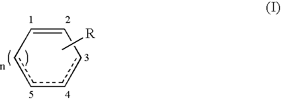
wherein
n is 1 or 3;
R is hydrogen or C₁-C₃ alkyl; and
i) if n is 1
the bond between C-3 and C-4 and C-5 and C-6 is a single bond and the dotted line together with the bond between C-4 and C-5 represents a double bond; or
the bond between C-4 and C-5 and C-5 and C-6 is a single bond and the dotted line together with the bond between C-3 and C-4 represents a double bond; and
ii) if n is 3
the bond between C-3 and C-4 and C-4 and C-5 is a single bond and the dotted line together with the bond between C-5 and C-6 represents a double bond; or
the bond between C-3 and C-4 and C-5 and C-6 is a single bond and the dotted line together with the bond between C-4 and C-5 represents a double bond; or
the bond between C-4 and C-5 and C-5 and C-6 is a single bond and the dotted line together with the bond between C-3 and C-4 represents a double bond;
or a mixture thereof.

2. Hydrocarbon gas according to claim 1 wherein the cycloalkadiene is selected from the group consisting of 1,5-cyclooctadiene, (Z,Z)-1,5-cyclooctadiene, (Z,E)-1,5-cyclooctadiene, 3-methyl-1,5-cyclooctadiene, 1,3-cyclohexadiene, and 1,4-cyclohexadiene.

3. A gas like odorant comprising
a) at least one cycloalkadiene of formula (I) as defined in claim 1; and
b) at least one sulfur-free compound selected from 1-methoxy-buten-3-yne, 2-methyl-1-buten-3-yne, cyclooctyne, pyrazines, and acrylic acid C₁-C₆ alkyl esters; and/or
c) at least one sulfur compound.

4. A hydrocarbon gas comprising a gas like odorant as defined in claim 3.

5. A method of odorizing hydrocarbon gas comprising the step of:
incorporating incorporation an effective amount of at least one cycloalkadiene of formula (I) as defined in claim 1 to the hydrocarbon gas.

6. A method of imparting a gas like odorant to a hydrocarbon gas comprising the step of: adding to the hydrocarbon gas an olfactorily effective amount of an odorant comprising the:
d) at least one cycloalkadiene of formula (I) as defined in claim 1;
e) and optionally further adding at least one sulfur-free compound selected from 1-methoxy-buten-3-yne, 2-methyl-1-buten-3-yne, cyclooctyne, pyrazines, and acrylic acid C₁-C₆ alkyl esters;
f) and optionally further adding at least one sulfur compound.

7. A method according to claim 6 wherein the cycloalkadiene is selected from the group consisting of: 1,5-cyclooctadiene, (Z,Z)-1,5-cyclooctadiene, (Z,E)-1,5-cyclooctadiene, 3-methyl-1,5-cyclooctadiene, 1,3-cyclohexadiene, and 1,4-cyclohexadiene.
